(19) United States
(12) Patent Application Publication
(10) Pub. No.: US 2023/0049584 A1
(43) Pub. Date: Feb. 16, 2023

(54) DEVICE FOR INSERTION INTO A CAVITY

(71) Applicant: AGILIS INTERNATIONAL LIMITED, Timaru (NZ)

(72) Inventor: Michael John RATHBONE, Hamilton (NZ)

(21) Appl. No.: 17/798,005

(22) PCT Filed: Feb. 9, 2021
(86) PCT No.: PCT/NZ2021/050015

§ 371 Date: Aug. 5, 2022

(30) Foreign Application Priority Data
Feb. 7, 2020 (NZ) 761513

(51) Int. Cl. A61M 31/00

(52) U.S. Cl. CPC A61M 31/002; A61M 2210/1475

(57) ABSTRACT

A device for insertion into a cavity, such as the vagina of a mammal, the device comprising a body for insertion into the cavity comprising a variable geometry retention mechanism, one or more active-impregnated matrices supported by the body, the matrix or matrices having (i) a ratio of exposed surface area to volume of 11.5:1 to 18.5:1, or (ii) a ratio of exposed surface area to weight of 11:1 to 17:1, or (iii) both (i) and (ii).

### FIELD OF THE INVENTION

The present invention relates to a device for insertion into a cavity, its method of manufacture and use.

### BACKGROUND TO THE INVENTION

Where artificial insemination is practiced, it may be useful for farmers to synchronise or control the onset of oestrus of the farmed production animals. These production animals may for example include mammals such as cattle, sheep, horses, pigs, goats, buffalo or deer.

Various means of bringing such production animals into oestrus are known. This includes, for example, the CIDR® and Cue-Mate® products for cattle. Further examples of these types of devices include US 4,449,980, WO 1997/040776, WO 1998/053758, WO 1999/063967, WO 1999/040966, WO 2001/012101, WO 2002/062415, WO 2017/117627, and Cappadoro and Luna “Development of an injection molded ethylene-vinyl acetate copolymer (EVA) intravaginal insert for the delivery of progesterone to cattle” (2015) 158: 104-108.

Such oestrus control devices operate by the diffusion of the active agent drug from the device to the animal, for absorption into the bloodstream of the animal. It is common for such devices to be intra-vaginally placed to release steroids or hormones such as progesterone for controlling the onset of oestrus.

The production and use of such products may involve significant costs. For example, material and production costs of the support structure and drug-carrying material may be considerable, such as where these involve the use of silicone materials. Furthermore, the drugs to be administered may be extremely expensive.

The control of oestrus may require specific targeted doses of a drug to be administered to an animal. For example, a desired blood level or progression of blood levels of the drug may be required to be achieved over a period of use of the device.

However, because of imperfect diffusion from the device, the dose of drug included in the device may be required to be greater than the total dose to be received by the animal. This will result in waste, as the device with quantities of the drug remaining in it are discarded after use. It may also increase the cost of the device both for a manufacturer to produce and a farmer to purchase, as it contains unused quantities of the drug. These cost problems may particularly be felt where the costs of the drugs, such as progesterone, are very high.

It is an object of the present invention to provide an intra-vaginal device (or insert), and methods of its manufacture and use that overcome deficiencies in the prior art devices, or which at least provide the public with a useful choice.

### SUMMARY OF THE INVENTION

In a first aspect, the invention relates to a device for insertion into a cavity, the device comprising
a body for insertion into the cavity comprising a variable geometry retention mechanism,
one or more active-impregnated matrices supported by the body, the matrix or matrices having
   i) a ratio of exposed surface area to volume of 11.5:1 to 18.5:1,
   ii) a ratio of exposed surface area to weight of 11:1 to 17:1, or
   iii) both (i) and (ii).

In a further aspect, the invention relates to a device for insertion into a cavity, the device comprising
a body for insertion into the cavity comprising a spine, a first wing, and second wing, one or more active-impregnated matrices, the first wing or the second wing, and
wherein a wing matrices, if present, has
   a surface area of approximately 25 cm² to approximately 35 cm², and
   a volume of approximately 2 cm³ to approximately 2.6 cm³ for a matrixes supported by a wing; and
wherein a spine matrices, if present, has
   a surface area of approximately 25 cm² to approximately 35 cm², and
   a volume of approximately 2 cm³ to approximately 2.6 cm³.

The following embodiments may relate to any of the above aspects.

Preferably the device is an intra-vaginal device.

Preferably the matrix has an exposed surface area of at least 145, 146, 147, 148, 149, 150, 151, 152, 153, 154 or 155 cm², and suitable ranges may be selected from between any of these values.

Preferably the one or more active-impregnated matrices have a volume of less than about 13.5, 13.4, 13.3, 13.2, 13.1, 13.0, 12.9, 12.8, 12.7, 12.6, 12.5, 12.4, 12.3, 12.2, 12.1 or 12.0 cm³, and suitable ranges may be selected from between any of these values.

Preferably the one or more active-impregnated matrices have a weight of less than about 14.0. 13.9. 13.8, 13.7, 13.6. 13.5, 13.4, 13.3, 13.2, 13.1, 13.0, 12.9, 12.8, 12.7, 12.6, 12.5, 12.4, 12.3, 12.2, 12.1 or 12.0 g, and suitable ranges may be selected from between any of these values.

Preferably the device contains 0.5, 0.6, 0.7, 0.8, 0.9, 1.0, 1.1, 1.2, 1.3, 1.4 or 1.5 g of one or more active agents, and suitable ranges may be selected from between any of these values.

Preferably the body has a weight of 9.5, 10.0, 10.5, 11.0, 11.5, 12.0, 12.5, 13.0 or 13.5 g, and suitable ranges may be selected from between any of these values.

Preferably the body has a volume of 7.0, 7.5, 8.0, 8.5, 9.0, 9.5, 10.0, 10.5, 11.0, 11.5 or 12.0 cm³, and suitable ranges may be selected from between any of these values.

Preferably the body has a surface area of about 75, 80, 85, 90 or 95 cm², and suitable ranges may be selected from between any of these values.

Preferably the active agent is a hormone selected from progesterone, estradiol, GnRH, prostaglandins and melatonin.

Preferably after insertion, the device maintains a plasma concentration of at least 2 ng/mL.

Preferably the active agent is an antibiotic selected from penicillins, cephalosporins, macrolides, fluoroquinolones, sulphonamides, tetracyclines, glycylcyclines, aminoglycosides, carbapenems, or a combination thereof.

Preferably the active agent is an anti inflammatory selected from for example non-steroidal anti-inflammatory drugs (NSAIDs), such as for example aspirin, ibuprofen, naproxen, aceclofenac, diclofenac, acetmetacin, dexibuprofen, ketoprofen, dexketoprofen, fenoprofen, flurbiprofen, etodolac, sulindac, indometacin, nebumetone, tiaprofenic acid, meloxicam, selective cyclo-oxygenase-2 (COX-2) inhibitors such as celecoxib, rofecoxib, etoricoxib or a combination thereof.

Preferably the active-impregnated matrices is over-moulded the body.

Preferably the active-impregnated matrices extend from the body. Preferably this extension is in the form of a flap. Preferably the flap is in the form of a sheet.

Preferably the active-impregnated matrices extend from at least two or more sides of the body.

Preferably the active-impregnated matrices extend laterally from the body.

Preferably the extensions of the active-impregnated matrices are substantially planar.

Preferably the active-impregnated matrices comprise an over-moulded region wherein the matrices coat an associated region of the body, and a peripheral projection from the associated region of the body.

In one embodiment the body comprises a body and at least one wing.

In one embodiment the spine carries one or more active-impregnated matrices and the spine has a ratio of surface area to volume of approximately 1:11 to 1:14, or 1:12 to 1:13, or more preferably about 1:12.8.

In one embodiment the active-impregnated matrices is carried by one or more wing elements, the wing matrices having a ratio of surface area to volume of about 1:11 to 1:15, or about 1:12 to 1:15, or about 1:13 to 1:14, or about 1:13.6.

In one embodiment the one or more active-impregnated matrices has a surface area of 25 cm² to 35 cm², and a volume of 2 cm³ to 2.6 cm³.

In one embodiment a first wing and a second wing each extend substantially opposite each other and laterally of the spine.

Preferably a portion of each of the first wing and second wing are resiliently flexible so as to provide a support structure of variable geometry to accommodate, variously, the insertion into, retention within, and removal of the device from the vagina of an animal.

Preferably the variable geometry retention mechanism is biased towards a retention condition.

Preferably at least one of the limbs of the support structure are of a substantially flat and elongate form.

Preferably at least a portion of the perimeter of one of the limbs being of a substantially flat and elongate form comprises one or more lateral recesses or lateral projections.

Preferably the moulding of the one or more active-impregnated matrices onto the one or more lateral recesses or lateral projections provides a material interference to the removal of the matrices from the limb.

Preferably at least a portion of the perimeter of one of the limbs being of a substantially flat and elongate form comprises a wave-like configuration.

Preferably the ratio of the surface area to volume of any or all of the over-moulding of the spine, the first wing, and the second wing is selected so as to provide both a) a pre-determined release rate of active agent, and b) a minimization of residual active within the over-moulding.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

The term “comprising” as used in this specification means “consisting at least in part of”. When interpreting statements in this specification which include that term, the features, prefaced by that term in each statement, all need to be present but other features can also be present. Related terms such as “comprise” and “comprised” are to be interpreted in the same manner.

### BRIEF DESCRIPTION OF THE DRAWINGS

The invention will now be described by way of example only and with reference to the drawings in which:

FIG. 1 shows the body of a device as described.

FIG. 2 shows the body of a device as described having an active-impregnated matrices supported by the spine of the body.

FIG. 3 shows a perspective view of a device having a matrix on by the spine and one of the wings.

FIG. 4 shows a perspective view of a device having an active-impregnated matrices on each of the two wings.

FIG. 5 shows a side view of Device B as used in the example.

FIG. 6 shows a top view of Device B as used in the example.

FIG. 7 is a graph showing changes in serum progesterone concentration after insertion of a Device A (CIDR) or Device B (test device) intravaginal progesterone releasing device. Solid circles represent the median with the 25^{th} and 75^{th} percentiles marked by vertical lines.

FIG. 8 is a graph showing the difference in serum progesterone concentration between cows receiving a Device A (CIDR) or Device B (test device) intravaginal progesterone releasing device.

### DETAILED DESCRIPTION OF THE INVENTION

The present invention relates to an intra-vaginal device. The components of the device (some of which may be formed unitary with other components of the device) include a body that comprises a variable geometry retention mechanism and at least one active-impregnated matrix held on the body.

The body may comprise a spine with separately attached wings that have variable geometry to assist retention of the device. Alternately, the body may be deigned in a manner that has variable geometry.

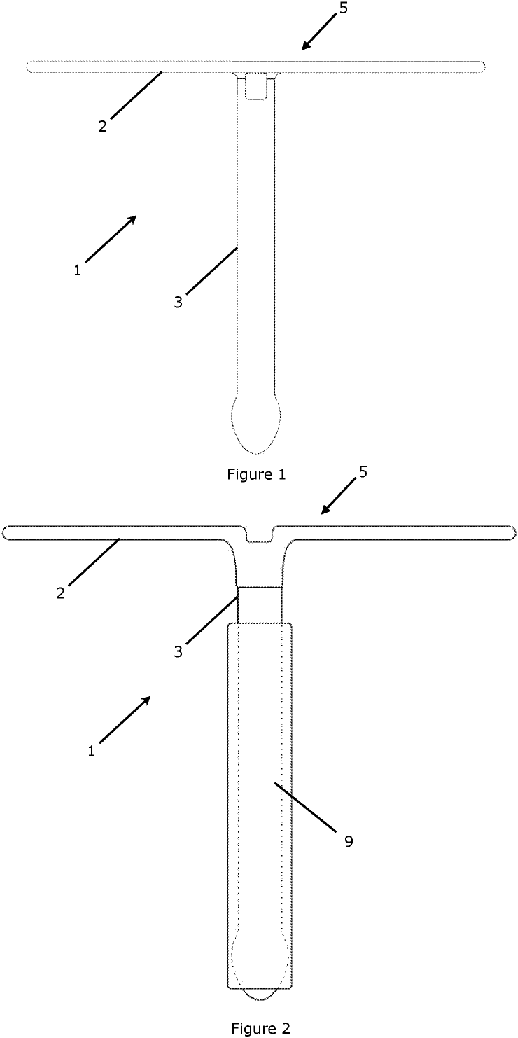

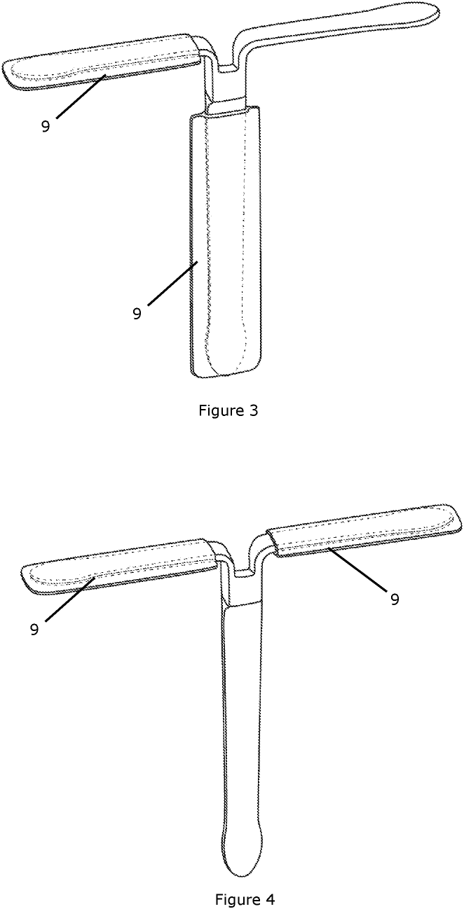

The active-impregnated matrix can be formed about, or attached to, any part of the body. For example, where the body comprises a central spine and wings, the active-impregnated matrix can be provided on the spine (see FIG. 2), a wing, or the spine and wing(s) (see FIG. 3). The matrices may be formed about, or attached to the wings of the wing retention mechanism as shown in FIG. 4.

The intra-vaginal device of the present invention can have a range of shapes depending on the arrangement of the particular components. For example, typical shapes of the intra-vaginal device include “T”’, “t”, “Y”, “y”, “V”, “C”, “X” or “M”-shaped, or wishbone, kite or diamond shaped.

Shown in Table 1 below are a range of intravaginal devices which demonstrate a range of different shapes. The wings of the “T”-shaped devices such as the CIDR inserts bend down against the spine of the insert upon insertion and flex outwardly once inserted to retain the device. The same effect is achieved from the other devices. For example, the arms (or wings) of the Cronipres Monodosis insert also have variable geometry allowing them to be bent against the body upon insertion to return to their “X”-shape to effect retention of the insert.

The “V”-shape of the Pulselar and Disposel inserts can be bent to an “I”shape for insertion where they then return to their “V”-shape to effect retention.

As can be seen, each of the devices includes a variable geometry to assist retention of the inserts in the cavity of the animal. The inserts may also include a cord attached to the insert that assists removal of the insert from the animal’s cavity.

TABLE 1

Other intravaginal devices

| Product | | Drug loading (g) | Body | | | Matrix | |
|---|---|---|---|---|---|---|---|
| | | | weight (g) | volume (cm³) | weight (g) | volume (cm³) | Surface area (cm²) |
| DIV-B | [image] | 1.0 | 9.4 | 7.0 | 29.5 | 26.0 | 131.2 |
| DIB-h | [image] | 0.5 | 9.4 | 7.0 | 29.5 | 26.0 | 130.5 |
| Pulselar | [image] | 1.0 | 9.3 | 7.0 | 29.4 | 26.0 | 129.9 |
| Pulselar 0.5 | [image] | 0.5 | 9.3 | 7.0 | 29.0 | 26.0 | 130.8 |
| Disposel Max | [image] | 1.2 | 11.1 | 9.0 | 24.7 | 23.0 | 128.5 |
| Disposel | [image] | 0.6 | 11.4 | 9.0 | 24.3 | 23.0 | 124.1 |
| D.I.V Rosenbusch | [image] | 0.6 | 11.3 | 10.0 | 23.0 | 21.4 | 134.7 |
| Cronipres Tres Udos | [image] | 1.0 | 11.3 | 10.0 | 29.7 | 26.0 | 143.8 |
| CIDR 1380 Cattle Insert | [image] | 1.38 | 12.4 | 10.6 | 14.8 | 13.8 | 126.3 |
| CIDR 1900 Cattle Insert | [image] | 1.9 | 11.6 | 10.0 | 19.1 | 18.0 | 131.2 |
| Repro One | [image] | 0.5 | 9.7 | 8.0 | 10.6 | 10.0 | 76.8 |
| Repro Neo | [image] | 1.0 | 15.5 | 13.0 | 24.8 | 23.4 | 160.9 |
| PRID Delta | [image] | 1.55 | 10.1 | 10.0 | 14.1 | 13.9 | 155.0 |
| Cronipres New | [image] | 0.5 | 11.8 | 10.0 | 20.2 | 19.0 | 92.3 |
| Cronipres Monodosis | [image] | 18.6 | 8.9 | 8.0 | 22.1 | 24.0 | 122.0 |
| Cue Mate | [image] | 1.6 | 9.3 | 8.0 | 28.4 | 28.0 | 160.6 |

### 1. Variable Geometry Retention Mechanism

The variable geometry retention mechanism 5 may take several forms, as shown in Table 1. As shown in FIGS. 1 to 4 the variable geometry retention mechanism may comprises at least two wing components 2 that extend laterally from the body’s spine 3. The purpose of the variable geometry retention mechanism 5 is to retain the device 1 in a cavity (such as the vaginal cavity) to prevent its uncontrolled ejection.

TABLE 2

Analysis of Other intravaginal devices

| Product | Ratio | | | |
|---|---|---|---|---|
| | Matrix | Matrix | Matrix | Matrix |
| | surface area to matrix volume | surface area to matrix weight | surface area to body volume | surface area to body weight |
| DIV-B | 5.0 | 4.4 | 32.8 | 28.9 |
| DIB-h | 5.0 | 4.4 | 32.6 | 28.3 |
| Pulselar | 5.0 | 4.4 | 32.5 | 28.2 |
| Pulselar 0.5 | 5.0 | 4.5 | 32.7 | 29.1 |
| Disposel Max | 5.6 | 5.2 | 32.1 | 30.7 |
| Disposel | 5.4 | 5.1 | 31.0 | 31.5 |
| D.I.V Rosenbusch | 6.3 | 5.9 | 19.5 | 18.4 |
| Cronipres Tres Udos | 5.5 | 4.9 | 24.0 | 19.3 |
| CIDR 1380 Cattle Insert | 9.2 | 8.5 | 16.2 | 15.9 |
| CIDR 1900 Cattle Insert | 7.3 | 6.9 | 13.1 | 12.8 |
| Repro One | 7.7 | 7.3 | 15.4 | 14.5 |
| Repro Neo | 6.9 | 6.5 | 13.6 | 12.9 |
| PRID Delta | 11.2 | 11.0 | 15.5 | 15.3 |
| Cronipres New | 4.9 | 4.6 | 9.2 | 7.8 |
| Cronipres Monodosis | 5.1 | 5.5 | 15.3 | 13.7 |
| Cue Mate | 5.7 | 5.7 | 20.1 | 17.3 |

When in use the wings 2 are bent down to be adjacent the spine 3 so that the device 1 can be inserted into a cavity. Once in position the wings 2 return to their resting position which means they push against the walls of the cavity holding the device 1 in place.

The wing 2 may be made of any material but is preferably made from plastic. The plastic may be degradable or non-degradable. The plastic may be a thermoplastic. For example, the frame may be formed from a polymer such as a polyethylene, polypropylene, starch-like polysaccarides such as Mater-Bi, polycaprolactone, poly glycolide, poly lactide, polyurethane, rubber, silicone, ethylene vinyl acetate, Nylon, or combinations thereof. Specific examples of suitable polymers include polyamide (Nylon), low-density polyethylene, high-density polyethylene, polypropylene, polycarbonate, polyester, polyacrylonitrile, polyolefin, acrylate such as polymethyl methacrylate), polyvinyl chloride, polyvinyl fluoride, polymethacrylate, high impact polystyrene, polystyrene, polysulfone, polybutadiene, polyaryletherketone, polyethylene terephthalate (PET), polyglycolic acid, polylatic acid, polylactic-co-glycolic acid, polycaprolactone, starches, polybutylene succinate, poly p-dioxanone, poly-3-hydroxybutyrate, or a combination thereof.

The wing surface provides a convenient surface for locating an active-impregnated matrix 9. As explained below, the active-impregnated matrix 9 may either be attached to the wing surface as an impregnated mass, or over-molded over the wing 2.

In one embodiment the intra-vaginal device 1 includes more than one variable geometry retention mechanism 5. This increases the total surface area that may support active agent loading.

Preferably, the active-impregnated matrix or matrices are over-molded onto the wing 2.

### 2. Spine

The spine 3 may be made of any material but is preferably made from plastic. The plastic may be degradable or non-degradable. The plastic may be a thermoplastic. For example, the frame may be formed from a polymer such as a polyethylene, polypropylene, starch-like polysaccarides such as Mater-Bi, polycaprolactone, poly glycolide, poly lactide, polyurethane, rubber, silicone, ethylene vinyl acetate, Nylon, or combinations thereof. Specific examples of suitable polymers include polyamide (Nylon), low-density polyethylene, high-density polyethylene, polypropylene, polycarbonate, polyester, polyacrylonitrile, polyolefin, acrylate such as polymethyl methacrylate), polyvinyl chloride, polyvinyl fluoride, polymethacrylate, high impact polystyrene, polystyrene, polysulfone, polybutadiene, polyaryletherketone, polyethylene terephthalate (PET), polyglycolic acid, polylatic acid, polylactic-co-glycolic acid, polycaprolactone, starches, polybutylene succinate, poly p-dioxanone, poly-3-hydroxybutyrate, or a combination thereof.

Preferably, the active-impregnated matrix 9 or matrices may be over-molded onto the spine 3.

In some embodiments, where the active-impregnated matrix 9 or matrices are over-molded onto the spine, the spine may have edges that are non-linear, for example, to better support a thin over-bolded layer of matrix. A wavy edge has been found to be particularly effective at supporting a thin matrix 9.

### 3. Active-Impregnated Matrix

The device 1 delivers an active agent to the cavity of a recipient animal.

In one embodiment, the device is an intra vaginal device that delivers the active agent to the vaginal cavity. Typically this is achieved by the presence of an active-impregnated matrix 9 that releases the active agent from the matrix over time.

The active-impregnated matrix 9 is typically formed of a polymer. The polymer may be a non-degradable thermoset polymer, a non-degradable thermoplastic polymer, a biodegradable polymer or a combination thereof. For example the polymer may be formed from silicone, ethylene vinyl acetate, polycaprolactone, natural (latex) rubber, styrene-butadiene rubber, polyurethane, polyamide (Nylon), low-density polyethylene (LDPE), high-density polyethylene (HDPE), polypropylene, polycarbonate, polyester, polyacrylonitrile, polyolefin, acrylic such as polymethyl methacrylate, polyvinyl chloride, polyvinyl fluoride, polymethacrylate, high impact polystyrene, polystyrene, polysulfone, polybutadiene, polyarylehetherketone, polyethylene terephthalate (PET), polyglycolic acid, polylactic acid, polyclatic-co-glycolic acid, starches, polybutylene succinate, poly p-dioxane, poly-3-hydroxybutyrate, or a combination thereof.

Preferably the active-impregnated matrix 9 or matrices is formed as a molding about the wing 2 or spine 3.

Preferably the impregnated mass is a molded sheet of silicon impregnated with an active agent.

In one embodiment the active-impregnated matrix is molded onto the wing 2 or spine 3. This technique is known and includes the process of placing the component (i.e. wing or spine) into a mold, injecting the active-impregnated matrix into the mold so that it forms a sheath around the component, for example as described in US 4,678,463.

Preferably the connection regions of the wing or spine are absent any molding.

Preferably the molding is overmolding of the wing 2 and/or spine 3.

A range of active agents could be utilised. For example the active agent may be selected from a natural or synthetic hormone, an antibiotic, antifungals or antivirals, a peptide or protein, an antiparasitic, an anti inflammatory, minerals, vitamins, trace elements, growth promotants, and any combination thereof.

As specific examples, where the active agent is a hormone, the hormone may be selected from progesterone, estradiol and testosterone.

Where the active agent is an antibiotic, it may be selected penicillins, cephalosporins, macrolides, fluoroquinolones, sulphonamides, tetracyclines, glycylcyclines, aminoglycosides, carbapenems, or a combination thereof.Where the active agent is an anti inflammatory it may be selected from for example non-steroidal anti-inflammatory drugs (NSAIDs), such as aspirin, ibuprofen, naproxen, aceclofenac, diclofenac, acetmetacin, dexibuprofen, ketoprofen, dexketoprofen, fenoprofen, flurbiprofen, etodolac, sulindac, indometacin, nebumetone, tiaprofenic acid, meloxicam, selective cyclo-oxygenase-2 (COX-2) inhibitors such as celecoxib, rofecoxib, etoricoxib or a combination thereof.

In one embodiment the, or each, active-impregnated matrix contain 1, 5, 10, 15, 20, 25, 30, 35, 40 45, 50, 55, 60, 65 or 75% by weight of the active agent, and useful ranges may be selected between any of these values (for example, about 1 to about 75, about 1 to about 60, about 1 to about 50, about 1 to about 40, about 1 to about 30, about 5 to about 75, about 5 to about 70, about 5 to about 65, about 5 to about 55, about 5 to about 40, about 5 to about 20, about 10 to about 75, about 10 to about 60, about 10 to about 50, about 10 to about 40, about 10 to about 30, about 15 to about 75, about 15 to about 65, about 15 to about 55, about 15 to about 25, about 20 to about 75, about 20 to about 60, about 20 to about 40, about 25 to about 75, about 25 to about 65, about 25 to about 60, about 25 to about 35, about 30 to about 75, about 30 to about 60, about 30 to about 40, about 35 to about 75, about 35 to about 65, about 35 to about 40, about 40 to about 75, about 40 to about 60, about 40 to about 50, about 45 to about 75, about 45 to about 60, about 50 to about 75, about 50 to about 70 or about 60 to about 75% by weight of the active agent).

In one embodiment the active-impregnated matrices comprise an over-moulded region wherein the matrices coat an associated region of the body, and a peripheral projection from the associated region of the body.

As shown in FIGS. 2 to 4, the active-impregnated matrices has an overlap portion that extends from the body. Preferably this extension is in the form of a flap and forms a sheet of matrix. As shown in these figures the active-impregnated matrices can extend from at least two or more sides of the body to form the extension flaps or sheets. Preferably they extend laterally from the body.

In one embodiment the extensions of the active-impregnated matrices are substantially planar. The active-impregnated matrix may be generally laminar and have substantially parallel faces.

The active-impregnated matrices may also include a release rate modifier that facilitates dissolution or retards diffusion of the active agent from the polymer. Examples of suitable release rate modifiers include inert fillers such as calcium carbonate, salts such as sodium chloride, pore forming substances such as water soluble salts, oils such as arachis and liquid paraffin, and surface active agents such as sodium lauryl sulphate.

In further preferred embodiments, the substance delivery device may be imbued with an active substance using processing methods such as compression molding, extrusion, casting or injection molding. One advantage of this method is that the manufacturing steps are less than the number required for injection molding of a traditional device as shown in New Zealand patent 207341 discussed above.

The ratio of the exposed surface area of the active-impregnated matrices to volume is important. For example, a larger value indicates a more efficient design of the device in terms of polymer (e.g. silicon) utilisation. For example, less polymer has been used to create a device with the required surface area. As a result of less polymer usage, and given the active agent is loaded into the device on a % w/w basis, less polymer therefore results in less active agent while achieving the required purpose.

For example, where the device is an intra-vaginal device, the device is designed to deliver an effective amount of drug, however with a lower amount of progesterone the result is less residual progesterone remaining in the device after use.

In one embodiment the ratio of exposed surface area of the drug impregnated matrix or matrices to the volume of the drug impregnated matrix or matrices is 11.5:1, 12:1, 12.5:1, 13:1, 13.5:1, 14:1, 14.5:1, 15:1, 15.5:1, 16:1, 16.5:1, 17:1, 17.5:1, 18:1 or 18.5:1, and suitable ranges may be selected from between any of these values, (for example, about 11.5:1 to about 18.5:1, about 11.5:1 to about 17.5:1,11.5:1 to about 16.5:1, about 11.5:1 to about 14.5:1, about 12:1 to about 18.5:1, about 12:1 to about 16:1, about 12:1 to about 14:1, about 12.5:1 to about 18.5:1, about 12.5:1 to about 16.5:1, about 12.5:1 to about 15:1, about 13:1 to about 18.5:1, about 13:1 to about 16:1, about 13:1 to about 15:1, about 13.5:1 to about 14:5, about 14:1 to about 18.5:1, about 14:1 to about 17:1, about 14:1 to about 16:1, about 14.5:1 to about 18.5:1, about 14.5:1 to about 17:1, about 15:1 to about 18.5:1).

In one embodiment the ratio of exposed surface area of the drug impregnated matrix or matrices to the weight of the drug impregnated matrix or matrices is 11:1, 12:1, 13:1, 14:1, 15:1, 16:1 or 17:1, and suitable ranges may be selected from between any of these values, (for example, about 11:1 to about 17:1, about 11:1 to about 16:1, about 11:1 to about 14:1, about 12:1 to about 17:1, about 12:1 to about 16:1, about 12:1 to about 14:1, about 13:1 to about 17:1, about 13:1 to about 15:1, about 14:1 to about 17:1).

In one embodiment the drug impregnated matrix or matrices has an exposed surface area of at least 145, 146, 147, 148, 149, 150, 151, 152, 153, 154 or 155 cm², and suitable ranges may be selected from between any of these values, (for example, about 145 to about 155, about 145 to about 153, about 145 to about 151, about 145 to about 150, about 145 to about 148, about 146 to about 155, about 146 to about 153, about 146 to about 151, about 146 to about 150, about 147 to about 155, about 147 to about 151, about 147 to about 150, about 148 to about 155, about 148 to about 154, about 148 to about 151, about 149 to about 155, about 149 to about 154, about 149 to about 151, about 150 to about 155 cm²).

In one embodiment the drug impregnated matrix or matrices have a volume of less than about 13.5, 13.4, 13.3, 13.2, 13.1, 13.0, 12.9, 12.8, 12.7, 12.6, 12.5, 12.4, 12.3, 12.2, 12.1 or 12.0 cm³, and suitable ranges may be selected from between any of these values, (for example, 13.5 to about 12. 13.5 to about 12.3, about 13.5 to about 12.5, about 13.5 to about 13.1, about 13.3 to about 12, about 13.3 to about 12.2, about 13.3 to about 12.6, about 13.3 to about 13.2, about 13.1 to about 12.0, about 13.1 to about 12.4, about 13.1 to about 12.8, about 13.0 to about 12.0, about 13.0 to about 12.2, about 13.0 to about 12.4, about 13.0 to about 12.6, about 12.9 to about 12.0, about 12.9 to about 12.2, about 12.9 to about 12.4, about 12.8 to about 12.0, about 12.8 to about 12.2, about 12.8 to about 12.4, about 12.7 to about 12.0, about 12.7 to about 12.2, about 12.7 to about 12.4 or about 12.6 to about 12.0 cm³).

In one embodiment the one or more active-impregnated matrices have a weight of less than about 14.0. 13.9. 13.8, 13.7, 13.6. 13.5, 13.4, 13.3, 13.2, 13.1, 13.0, 12.9, 12.8, 12.7, 12.6, 12.5, 12.4, 12.3, 12.2, 12.1 or 12.0 g, and suitable ranges may be selected from between any of these values, (for example 14 to about 12, about 14 to about 12.5, about 14 to about 13, about 13.5 to about 12, about 13.5 to about 12.1, about 13.5 to about 12.5 or about 13 to about 12 g).

In one embodiment the device contains 0.5, 0.6, 0.7, 0.8, 0.9, 1.0, 1.1, 1.2, 1.3, 1.4 or 1.5 g of one or more active agents, and suitable ranges may be selected from between any of these values, (for example, about 0.5 to about 1.5, about 0.5 to about 1.3, about 0.5 to about 1.0, about 0.6 to about 1.5, about 0.6 to about 1.4 0.6 to about 1.2, about 0.6 to about 1.0, about 0.7 to about 1.5, about 0.7 to about 1.3, about 0.7 to about 1.0, about 0.8 to about 1.5, about 0.8 to about 1.3, about 0.8 to about 1.1, about 0.9 to about 1.5, about 0.9 to about 1.3 or about 0.9 to about 1.2 g).

In one embodiment the body has a weight of 9.5, 10.0, 10.5, 11.0, 11.5, 12.0, 12.5, 13.0 or 13.5 g, and suitable ranges may be selected from between any of these values, (for example 9.5 to about 13.5, about 9.5 to about 12.5, about 9.5 to about 11.5, about 10 to about 13.5, about 10 to about 12, about 10.5 to about 13.5, about 10.5 to about 12, about 11 to about 13.5, about 11 to about 12, about 11.5 to about 13.5, about 11.5 to about 13, about 11.5 to about 12.5, about 12 to about 13.5 g).

In one embodiment the body has a volume of 7.0, 7.5, 8.0, 8.5, 9.0, 9.5, 10.0, 10.5, 11.0, 11.5 or 12.0 cm³, and suitable ranges may be selected from between any of these values, (for example 7 to about 12, about 7 to about 11.5, about 7 to about 11, about 7 to about 10, about 7 to about 9, about 8 to about 12, about 8 to about 11, about 9 to about 12, about 9 to about 11 or about 10 to about 12 cm³).

In one embodiment the body has a surface area of about 75, about 80, about 85, about 90 or 95 cm², about and suitable ranges may be selected from between any of these values, about (for example 75 to about 95, about 75 to about 90, about 75 to about 85, about 80 to about 95, about 80 to about 90, about 85 to about 95 cm²).

### 4. Manufacture of the Device

In one embodiment the invention is a method of manufacturing a device comprising providing a body having a variable geometry retention mechanism. Preferably the body is comprised of a spine that supports the variable geometry retention mechanism. As state earlier, preferably the variable geometry retention mechanism is formed from one or more wings that depend from the body. The active impregnated matrix is then over-moulded about a portion of the body, such as the spine and/or wing.

In one embodiment the body of the device 1 made up of one or more wings 2 and a spine 3 that can be manufactured through normal methods such as extrusion and moulding.

Typically the spine will have some means to attach a withdrawal string or filament that aids withdrawal of the device from the animal.

The active-impregnated matrix is formed by mixing an active agent with the polymer.

The active-impregnated matrices is preferably manufactured using compression or injection molding over-moulded a portion of the body.

Where compression molding is used, the active agent is first mixed with the polymer. For example, 1, 5, 10, 15, 20, 25, 30, 35, 40, 45, 50 or 60% by weight of active agent is fixed with the polymer, and useful ranges may be selected between any of these values (for example, about 1 to about 60, about 1 to about 45, about 1 to about 25, about 1 to about 20, about 5 to about 60, about 5 to about 40, about 5 to about 30, about 5 to about 20, about 5 to about 15, about 10 to about 60, about 10 to about 50, about 10 to about 40, about 10 to about 25, about 10 to about 20, about 15 to about 60, about 15 to about 35, about 15 to about 25, about 20 to about 60, about 20 to about 50, about 20 to about 40, about 25 to about 60, about 25 to about 45, about 25 to about 35, about 30 to about 60, about 30 to about 50, about 30 to about 40, about 35 to about 60, about 35 to about 45, about 40 to about 60, about 40 to about 50, about 45 to about 60, about 45 to about 50 or about 50 to about 60% by weight).

The mixture of active and polymer is then mixed. Preferably the active and polymer are mixed by rollers.

The body is placed into a mold cavity along. It will be appreciated that the size and shape of the mould cavity is designed to produce the active-impregnated matrices having the desired dimensions over moulded over the body of the device. An amount of active/polymer mixture is then introduced into the mold cavity.

The active/polymer mixture placed into the mould is heated to about 50, 60, 80, 100, 120, 140, 160, 180, 200, 220, 240, 260, 280 or 300° C., and useful ranges may be selected between any of these values under pressure of about 150, 300, 450, 600, 750, 900, 1050, 1200, 1350, 1500, 1650, 1800, 1950, 2100, 2250, 2400, 2550, 2700, 2850 or 3000 psi until the active/polymer mixture has polymerized and cured.

Where injection molding is used, the active agent is first mixed with the polymer. For example, 1, 5, 10, 15, 20, 25, 30, 35, 40, 45, 50 or 60% by weight of active agent is fixed with the polymer, and useful ranges may be selected between any of these values (for example, about 1 to about 60, about 1 to about 45, about 1 to about 25, about 1 to about 20, about 5 to about 60, about 5 to about 40, about 5 to about 30, about 5 to about 20, about 5 to about 15, about 10 to about 60, about 10 to about 50, about 10 to about 40, about 10 to about 25, about 10 to about 20, about 15 to about 60, about 15 to about 35, about 15 to about 25, about 20 to about 60, about 20 to about 50, about 20 to about 40, about 25 to about 60, about 25 to about 45, about 25 to about 35, about 30 to about 60, about 30 to about 50, about 30 to about 40, about 35 to about 60, about 35 to about 45, about 40 to about 60, about 40 to about 50, about 45 to about 60, about 45 to about 50 or about 50 to about 60% by weight).

The mixture of active and polymer is then mixed. Preferably the active and polymer are mixed by the use of a bladed stirrer.

A mold is selected that is of a size and shape such that the inner chamber matches to the desired dimensions of device having the active-impregnated matrices over-moulded the body. The body is intorduced into the mould, the two halves of the mold are clamped together and the active/polymer mixture injected into the mold. The mold is heated to cure the active/polymer mixture.

### 5. Use of the Device

The present device can be insered into a cavity where it is retained in the cavity by the variable geometry mechanism. Once in the cavity the drug impregnated in the matrix or matrices leaches out of the matrix.

More particularly the device can be inserted into the vaginal cavity of a mammal. For example, to control estrus in a mammal through the release of progesterone from the matrix or matrices.

Therefore, in one embodiment the method relates to the intra-vaginal administration of an active agent to an animal by providing or assembling an intravaginal device as described. The intravaginal device comprising a body that supports a variable geometry retention mechanism (e.g. such as one or more wing elements that depend from the body) and one or more active impregnated matrixes that are over-moulded over a portion of the body (such as the spine and/or the wing elements).

The device of the present invention can be used for administering an active agent to mammals, including domesticated animals such as cattle, heifers, sheep, camels, pigs and goats.

Given the present devices are designed to deliver a similar amount of active as known devices, their ability to incorporate less active into the matrices, means that there is less residual progesterone remaining in the device’s matrices after use. Therefore, less active agent and silicon is discarded into the environment after use.

In one embodiment the delivery device achieves an active agent release rate of at least about 0.1, 0.15, 0.2, 0.25, 0.3, 0.35, 0.4, 0.45, 0.5, 0.55 or 0.6 mg.cm².day¹, and useful ranges may be selected between any of these values, (for example, 0.1 to about 0.6, about 0. to about 0.55, about 0.1 to about 0.4, about 0.1 to about 0.3, about 0.15 to about 0.6, about 0.15 to about 0.5, about 0.15 to about 0.4, about 0.2 to about 0.6, about 0.2 to about 0.5, about 0.2 to about 0.4, about 0.2 to about 0.35, about 0.25 to about 0.6, about 0.25 to about 0.55, about 0.25 to about 0.4. 0.3 to about 0.6, about 0.3 to about 0.5, about 0.35 to about 6, about 0.35 to about 0.45, about 0.4 to about 0.6, about 0.4 to about 0.55, about 0.45 to about 0.6 mg.cm².day¹).

In one embodiment the delivery device is used to control estrous in an animal. The control of estrous typically requires the device to achieve a sufficient blood plasma concentration of progesterone. For example, in bovine animals a blood plasma concentration of at least 2 ng/mL is desired to be maintained to effectively control estrous. In pigs a blood plasma concentration of at least 8-10 ng/mL is desired to control estrous.

One aspect of the invention relates to a method of regulating the estrous cycle of an animal comprising inserting the delivery as described into a body cavity of the animal and then after a period of time, withdrawing the device. Preferably the delivery device is inserted into the vaginal cavity of the animal to control estrous, wherein the device releases progesterone upon insertion.

Preferably, when in an assembled form, the intra-vaginal device can form to a deployed and undeployed condition. The shape of the drug delivery device is such to fit easily and comfortably inside an animal’s vagina. The impregnated mass of the drug delivery device is imbued with sufficient active agent (such as progesterone) to provide exogenous active agent (i.e. the progesterone) sufficient to have a biological effect (e.g. to control the estrous cycle in the case of progesterone). The drug delivery device may also have a cord to facilitate removal after the desired administration period

The device of the present invention can be used for administering an active agent to mammals, including domesticated animals such as cattle, heifers, sheep, camels, pigs and goats.

Preferably the desired drug loading of the intra-vaginal device is determined from the weight of the animal.

The active agent delivered may include natural or synthetic hormones, an antibiotic, antifungals or antivirals, a peptide or protein, an antiparasitic, an anti inflammatory, minerals, vitamins, trace elements, growth promotants, and any combination thereof.

Examples of substances that can be incorporated into the substance delivery device include, but are not limited to, drugs used in estrous control such as natural or synthetic hormones including progesterone, estradiol, GnRH, prostaglandins, melatonin, drugs used to control infections such as antibiotics, antifungals or antivirals, drugs such as proteins and peptides, drugs used to control parasitic infections including anthelmintics such as ivermectin, abermectin, oxfendazole, macrolytic lactones, oxfendazole or levamisole, nutrients such as minerals, vitamins, trace elements, growth promotants.

For intravaginal devices the preferred active agent includes natural or synthetic hormones.

Drug release from the substance delivery device is influenced by the surface area, thickness of the silicone or drug load. Thus the rate (amount per day), extent (total amount released or drug utilization) and duration (length of treatment) can be predetermined. In addition the substance delivery device may be made with different polymers, with each different polymer being attached to the same plastic shape of proper design, thereby permitting different release rates of different drugs, either simultaneously or sequentially from the same device.

### EXAMPLE - EQUIVALENCE STUDY

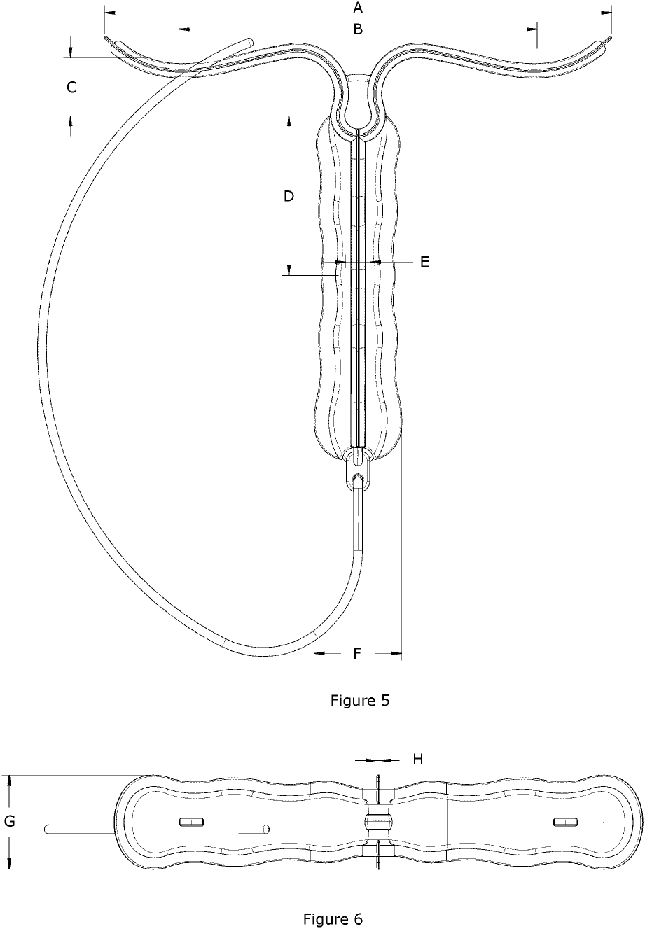

This study was undertaken to investigate the equivalence between the two products: (A) CIDR or (B) intra-vaginal progesterone releasing implant as shown in FIGS. 5 and 6.

Device B comprised a typical silicone thickness of 0.8 mm and had a total silicone (matrix) exposed area of 158.60 cm². The total silicone (matrix) volume was 19.1 cm³. The spine and tail material was Nylon PA66. Device B comprised the following measurements:

| | |
|---|---|
| a) 144.4 mm | e) 7 mm |
| b) 102 mm | f) 25 mm |
| c) 16.5 mm | g) 25.8 mm |
| d) 45.5 mm | h) 0.8 mm |

The study comprised 25 cows in each group. The cows were treated with prostaglandin F2a 6 days and 4 days before device insertion. The intravaginal devices were inserted for 10 days and released progesterone. The sequential daily serum progesterone samples from the 50 cows were measured at repeated intervals, from day 0 to day 12.

As shown in Table 3 are the pre-defined measures for equivalence. Results falling within these pre-defined measurements shows equivalence between the two devices tested.

TABLE 3

Pre-defined measure of equivalence

| | | |
|---|---|---|
| Dependent variable | Region of probable equivalence for comparison of means | Region of probable equivalence for comparison of data |
| Cmax | +/- 0.5 ng/mL | +/- 1.5 ng/mL |
| AUC | +/- 50 ng/mL x hours | +/- 150 ng/mL x hours |
| Tmax | +/- 24 hours | +/- 24 hours |
| Serial progesterone concentration at time t | +/-0.2 ng/mL | +/- 2 ng/mL |

TABLE 4

Summary of calculated variables by device

| dep.var | device | n | mean | sd | median | min | max |
|---|---|---|---|---|---|---|---|
| Cmax (ng/mL) | A | 25 | 4.90 | 3.02 | 3.5 | 2.7 | 15.4 |
| Cmax (ng/mL) | B | 25 | 4.55 | 2.85 | 3.4 | 2.5 | 15.8 |
| Tmax (hours) | A | 25 | 78.3 | 78.1 | 72 | 2 | 212 |
| Tmax Hours) | B | 25 | 53.6 | 55.57 | 48 | 2 | 212 |
| AUC (hours x ng/mL) | A | 25 | 653.8 | 231.77 | 558.9 | 405.6 | 1346.2 |
| AUC (hours x ng/mL) | B | 25 | 620.9 | 268.29 | 532.3 | 453.6 | 1530.5 |

Two levels of analysis were conducted: analysis of mean Cmax, Tmax and AUC by group and analysis of mean serum progesterone concentration by device and time.

### Mean Cmax for Cows Receiving Device B or CIDR

The mean difference in serum progesterone Cmax between cows receiving a CIDR and those receiving Device B was -0.26 (95%PI=-0.264 — -0.248) ng/mL. This is well within the ROPE (region of probable equivalence) (+/-0.5 ng/mL). We are 95% certain that on average, the mean serum progesterone Cmax will be 0.26 ng/mL lower in cows receiving a Device B than cows receiving a CIDR. However, the narrow predictive interval suggests this mean difference is reliably small.

### Range of Cmax Values for Cows Receiving Device B or CIDR

The average difference comes from a population of differences so it is important to understand what the range of differences could be. With an average difference of -0.26 ng/mL, 95 percent of the differences in serum progesterone Cmax between individual cows receiving a CIDR and those receiving a Device B ranged from -1.3 to 0.77 ng/mL. Thus the predicted differences are well within the ROPE of +/-1.5 ng/mL suggesting that we are more than 95% confident that the difference in serum progesterone Cmax between a cow receiving a Device B and one receiving a CIDR will be less than +/- 1.5 ng/mL.

### Mean Area Under the Curve for Cows Receiving Device B or CIDR

The difference in mean serum progesterone AUC between cows receiving a CIDR and those receiving a Device B was -35.7 (95%PI=-36.5 — -35.0) ng/mL. This is well within a ROPE of +/-50 ng/mL-hour. Thus, we are 95% certain that on average, the mean serum progesterone AUC will be 35.7 ng/mL hours lower in cows receiving a Device B than cows receiving a CIDR. However, the narrow predictive interval suggests this mean difference is reliably small.

### Range of Area Under the Curve for Cows Receiving Device B or CIDR

With an average difference of -35.7 ng/mL-hour, 95 percent of the differences in serum progesterone AUC between cows receiving a CIDR and those receiving a Device B ranged from -142.4 to 70.5 ng/mL-hour, with an average difference of -35.3. The predicted differences are well within the ROPE of +/-150ng/mL-hour suggesting that we are more than 95% confident that the difference in serum progesterone AUC between a cow receiving a Device B and one receiving a CIDR will be less than +/- 150 ng/mL-hour.

### Mean Tmax for Cows Receiving Device B or CIDR

The mean difference in Tmax between cows receiving a CIDR and those receiving a Device B was -21 (95%PI=-21.2 — -20.8) ng/mL-hour. This is well within the ROPE (+/-24-hour). We are 95% certain that on average, the mean Tmax will be 21 hours earlier in cows receiving a Device B than cows receiving a CIDR. However, the narrow predictive interval suggests this mean difference is reliably around 21 hours.

### Range of Tmax Values for Cows Receiving Device B or CIDR

With an average difference of -21 hours, 95 percent of the differences in Tmax between cows receiving a CIDR and those receiving a Device B ranged from -52 to +6 hours. Only 52% of the predicted values are within the ROPE (+/-24 hours) suggesting that there is evidence for a difference in Tmax between cows receiving a CIDR and those with a Device B, with Device B cows consistently developing Cmax 21 hours before CIDR cows.

### Changing Concentrations of Serum Progesterone with Time

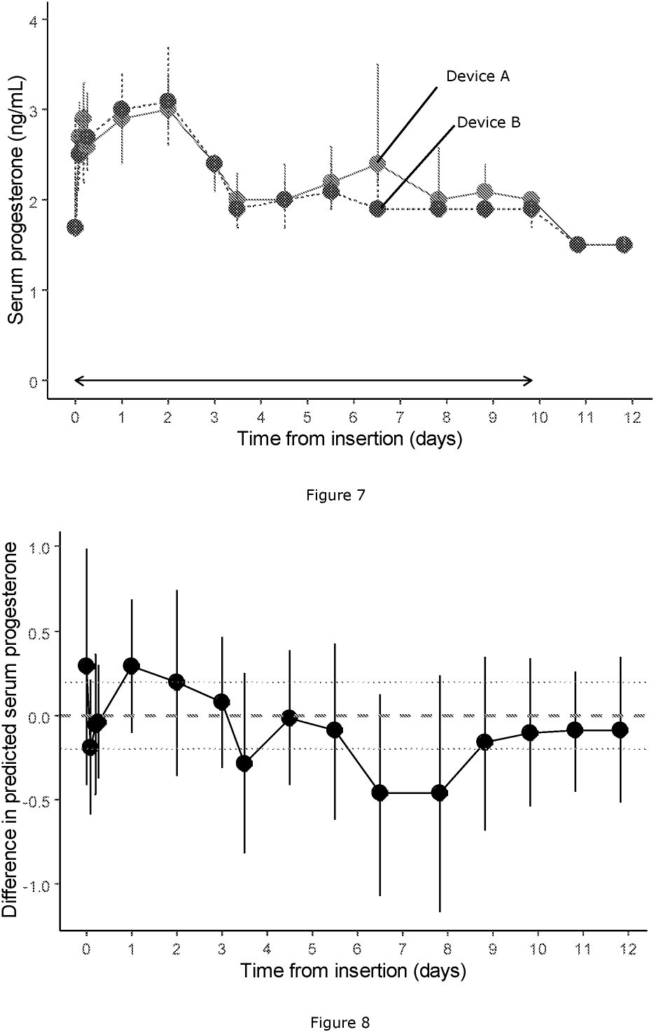

The repeated measures of serum progesterone for cows receiving a CIDR or a Device B are shown in FIG. 7.

FIG. 12 shows the difference in serum progesterone concentration between cows receiving a CIDR or a Device B intravaginal progesterone releasing device. Solid circles represent the median with the 25th and 75th percentiles marked by vertical lines. The upper and lower limit of the ROPE (+/- 0.2 ng/mL) marked by dashed red lines.

This suggests that on a sequential basis the average median concentration of serum progesterone is within 0.2 ng/mL for cows treated with either a CIDR or a Device B, although the wide inter-quartile range means that the difference between treatments exceeds the ROPE in some cows.

### Median Overall Serum Progesterone Concentration During Insertion for Cows Receiving Device B or CIDR

The difference in median serum progesterone between cows receiving a CIDR and those receiving a Device B over ten days following insertion was 0.08 (95%PI=0.07 – 0.09) ng/mL. This is well within the ROPE (+/-0.2 ng/mL).

### Range in Serum Progesterone Concentration During Insertion for Cows Receiving Device B or CIDR

With a median difference of 0.07 ng/mL, 95 percent of the differences in serum progesterone measured in the 10 days following insertion between cows receiving a CIDR and those receiving a Device B ranged from -3 to +2.8 ng/mL. 90% of the estimated values are within the ROPE of +/- 0.2 ng/mL.

Within a range of limits of equivalence, there was no difference in the average or range of maximum progesterone concentration (Cmax), area under the progesterone concentration curve (AUC), daily progesterone concentration or overall progesterone concentration during the study period between cows treated with either device.

Tmax was on average 21 hours earlier (05%PI = 20.8-21.2) for cows receiving a Device B and 48% of animals treated with a Device B experienced Tmax between 21 and 52 hours before cows treated with a CIDR.

Aspects of the present invention have been described by way of example only and it should be appreciated that modifications and additions may be made thereto without departing from the scope of the patent claims.

1. A device for insertion into a cavity, the device comprising:
a body for insertion into the cavity comprising a variable geometry retention mechanism; and
one or more active-impregnated matrices over-moulding at least a portion of the body and extending from the body to form a flap, the one or more active-impregnated matrices having:
i) a ratio of exposed surface area to volume of 11.5:1 to 18.5:1,
ii) a ratio of exposed surface area to weight of 11:1 to 17:1, or
iii) both (i) and (ii).

2. The device of claim 1, wherein the device is an intravaginal device.

3. A-The device of claim 1, wherein the active-impregnated matrices has an exposed surface area of at least 145 to 155 cm².

4. The device of claim 1, wherein active-impregnated matrices have a volume of less than about 13.5 to 12.0 cm³.

5. A-The device of claim 1, wherein the active-impregnated matrices have a weight of less than about 14.0 to 12.0 g.

6. The device of claim 1, wherein the device contains 0.5 to 1.5 g of one or more active agents.

7. A-The device of claim 1, wherein the body has a weight of 9.5 to 13.5 g.

8. The device of claim 1, wherein the body has a volume of 7.0 to 12.0 cm³.

9. The device of claim 1, wherein the body has a surface area of about 75 to 95 cm².

10. The device of claim 1, wherein the device maintains a plasma concentration of at least 2 ng/mL.

11-13. (canceled)

14. The device of claim 1, wherein the active-impregnated matrix or matrices extend from at least two or more sides of the body.

15. The device of claim 1, wherein the active-impregnated matrix or matrices extend laterally from the body.

16. The device of claim 1, wherein the extensions of the active- impregnated matrix or matrices are substantially planar.

17. The device of claim 1, wherein at least a portion of the perimeter of the body is of a substantially flat and elongate form comprising a wave-like configuration.

18. The device of claim 1, wherein the ratio of the surface area to volume of any or all of the over-moulding of the spine, the first wing, and the second wing is selected so as to provide both a) a pre-determined release rate of active agent, and b) a minimization of residual active within the over-moulding.
